(19) United States
(12) Patent Application Publication
(10) Pub. No.: US 2022/0404365 A1
(43) Pub. Date: Dec. 22, 2022

(54) METHODS FOR DETERMINING THE INVASIVE AND/OR METASTATIC POTENTIAL OF A TUMOUR

(71) Applicant: University of Bradford, Bradford (GB)

(72) Inventor: Mohamed El-Tanani, Amman (JO)

(21) Appl. No.: 17/755,279

(22) PCT Filed: Oct. 27, 2020
(86) PCT No.: PCT/GB2020/052718

§ 371 Date: Apr. 26, 2022

(30) Foreign Application Priority Data
Oct. 28, 2019 (GB) 1915598.5

(51) Int. Cl. G01N 33/574; C12Q 1/6886

(52) U.S. Cl. CPC G01N 33/57415; C12Q 1/6886; G01N 33/5748; C12Q 2600/118; C12Q 2600/158; G01N 2333/96494

(57) ABSTRACT

The invention provides methods for determining tumour status in a subject comprising the steps of: (i) determining a quantitative value in a sample taken from a subject of a first biomarker selected from the group consisting of Ran, Ran binding protein 1, an active fragment of a Ran protein, a nucleic acid sequence encoding Ran, a nucleic acid sequence encoding Ran binding protein 1, a nucleic acid sequence encoding an active fragment of Ran and a nucleic acid sequence encoding an active fragment of Ran binding protein 1; (ii) comparing the quantitative value of the first biomarker in the sample with a selected pre-determined threshold value of the first biomarker; (iii) determining a quantitative value in a sample from the same subject of a second biomarker selected from the group consisting of MMP2, an active fragment of MMP2, a nucleic acid sequence encoding MMP2 and a nucleic acid sequence encoding an active fragment of MMP2; (iv) comparing the quantitative value of the second biomarker in the sample with a selected pre-determined threshold value of the second biomarker; wherein the quantitative values of the first marker and the second biomarkers in the sample as compared to their respective selected pre-determined threshold values indicate whether or not the tumour sample has invasive and/or metastatic potential.

The present invention is generally concerned with methods for determining the risk of metastasis of a cancer of the human or animal body. The present invention is particularly, but not exclusively, concerned with methods for determining the metastatic and/or invasive potential of a tumour from a sample taken from the human or animal body. However, it also is concerned with a method for determining the risk of metastastis and metatstatic potential from a sample and monitoring metastatic potential taken from the human or animal body following surgery removing the tumour.

RAN, a member of the RAS Oncogene family, is a gene that encodes the GTP-binding nuclear protein Ran. Analysis of the GEO breast cancer data set of (200 patients (GSE2034)) indicated that a high level of Ran significantly correlates to shorter survival time in patients with PIK3CA mutation gene signature (P=0.018), but not in those with PIK3CA wild-type gene signature (P=0.186); Yuen H-F. et al. in J Natl Cancer Inst. 2013, 105, 475-488 and Yuen H-F. et al. in Clin Cancer Res. 2012, 18, 380-391). Breast cancer patients whose primary tumours have a higher percentage of malignant cell nuclei that stain for Ran have a shorter median survival time than those with less than 1% of cell nuclei that stain for Ran (P<0.001) (Yuen H-F. et al. in J Natl Cancer Inst. 2013, 105, 475-488 and Yuen H-F. et al. in Clin Cancer Res. 2012, 18, 380-391). It is also known that higher levels of Ran are significantly correlated with a shorter survival time in lung cancer patients. Overexpression of RAN gene is observed in a number of cancers and this overexpression has been linked to poor patient prognosis. For example, RAN overexpression has been shown to correlate with increased aggressiveness of cancer cells in vitro and in vivo (Kurisetty W. et al, in Oncogene 2008, 27(57), 7139-7149), i.e. RAN overexpressing cancer cells are seen to grow rapidly and exhibit high metastatic potential. In contrast, silencing RAN by siRNA or shRNA reduced cell adhesion, migration and invasion in vitro and metastasis in vivo. Furthermore, one of the inventors of the present invention has described, in EP2082225B1, the use of an assay based upon the overexpression of RAN in cancer cells for the prediction of survival of cancer patients.

A number of other proteins which are known to be overexpressed in human cancers include c-Myc, c-Met and MMP2. c-Myc is a human proto-oncogene which plays an important role in reglating cell growth. MMP2 (matrix metalloproteinase-2) is type IV collagenase that is involved in the breakdown of extracellular matix (ECM) in normal physiological processes. Altered expression and activity levels of MMPs have been strongly implicated in the progression and metastasis of many forms of cancer. Increased MMP2 activity has been linked to poor prognosis in multiple forms of cancer (Norklund M. and Koivunen E. in Bichimica et Biophysica Acta 2005). It is also known that each of Ran, c-Met, c-Myc and MMP2 are independently significantly associated with patient demise from metastatic breast cancer.

A number of assays (Oncogene DX®, Prosigna® and EndoPredict®) for predicting the risk of metastasis in oestrogen receptor positive and human epidermal growth factor receptor 2 negative (ER +ve/HER2 −ve) breast cancers have been approved for clinical use in the UK. These assays, which are not approved for use with any other cancer, or indeed any other breast cancer sub-type, are based on the determination of levels of a host of genes within a ER +ve/HER2 −ve tumour cell and comparison with levels within an historical patient cohort of ER +ve/HER2 −ve breast cancer patients which are statistically correlated with patients not developing metastasis.

This correlation, which is expressed as a negative percentage response (NPR) may provide a high degree of confidence that a patient will not go on to develop metastasis and may mean that a patient can avoid such debilitating cancer treatments as surgery, chemotherapy or radiotherapy.

Notwithstanding that the NPR for these assays are higher than 90% and even about 95%, there exists a need for an improved assay for predicting the risk of metastasis in ER +ve/HER2 −ve breast cancer patients. There also exists a need for a reliable assay for predicting the risk of metastasis in other cancer patients, particularly in patients with breast cancers other than ER +ve/HER2 −ve breast cancer or patients with ovarian, colon or lung cancer.

An assay offering even a slightly higher NPR may be highly significant for a patient diagnosed with ER +ve/HER2 −ve breast or other cancer, in that the knowledge that the cancer does not have invasive and/or metastatic potential may mean that the patient can avoid debilitating cancer treatments such as surgery, chemotherapy or radiotherapy, which that patient might otherwise have undertaken.

The present invention resides in the new findings that Ran is involved in a cell-signalling pathway containing c-Met, c-Myc and MMP2 and that this is linked to increase in the metastatic properties of cultured cells. Inventors have also found that levels of Ran, c-Met, c-Myc and MMP2 in the tumours of breast cancer patients who are at high risk of dying from metastatic disease are increased by between 5 fold and 10 fold as compared to levels of c-Met, c-Myc and MMP2 in the tumours of breast cancer patients who are at low risk of dying from metastatic disease. It has further been found that patient survival in breast cancers involving Ran overexpression is better correlated with levels of Ran and MMP2 than with the level of Ran alone.

Accordingly, the present invention advantageously provides simple assays which can determine tumor status, including predicting the risk of metastasis not just for ER +ve/HER2 −ve breast cancer but also for other cancers, including other sub-types of breast cancer. The assays may provide a NPR greater than or equal to 97%, for example, 97.5% or 98% or even more (any and decimal numerical therebetween) in all breast cancer sub-types.

In a first aspect of the present invention there is provided a method for determining tumour status in a subject comprising the steps of:
(i) determining a quantitative value in a sample taken from a subject of a first biomarker selected from the group consisting of Ran, Ran binding protein 1, an active fragment of a Ran, a nucleic acid sequence encoding Ran, a nucleic acid sequence encoding Ran binding protein 1 or derivative thereof, a nucleic acid sequence encoding an active fragment of Ran or a derivative thereof and a nucleic acid sequence encoding an active fragment of Ran binding protein 1;
(ii) comparing the quantitative value of the first biomarker in the sample with a selected pre-determined threshold value of the first biomarker;
(iii) determining a quantitative value in a sample of a second biomarker selected from the group consisting of MMP2, an active fragment of MMP2, a nucleic acid sequence encoding MMP2 or a derivative thereof, a nucleic acid sequence encoding an active fragment of MMP2 or a derivative thereof and a nucleic acid encoding an active fragment of MMP2 protein;
(iv) comparing the quantitative value of the second biomarker in the sample with a selected pre-determined threshold value of the second marker;
wherein the quantitative values of the first biomarker and the second biomarker in the sample as compared to their respective selected pre-determined threshold values indicate whether or not the tumour sample has invasive and/or metastatic potential.

Reference herein to “tumour status” includes an initial diagnosis, monitoring for metastasis following surgery and/or during chemotherapy or radiotherapy, stratification of a group of subjects with cancer and/or predicting probability of survival.

Reference herein to “quantitative value” includes the number of cells expressing the first and second biomarkers in a sample obtained from a subject, the level of cells expressing the first and second biomarkers in a biological sample obtained from a sample or any other methodology which is capable of ascertaining a quantitative value/level of the first and second biomarkers in a biological sample obtained from the subject.

A “biological sample” is a biological sample or biological material likely to contain both the first and second biomarkers. The biological material which may be derived from any biological source that is removed from the cancer patient by standard methods which are well-known to a person having ordinary skill in the art

Reference herein to “sample” includes a sample obtained from any one or more of the following sources: a solid tumour biopsy, a liquid tumour biopsy, a tumour cell, circulating tumour cells in blood and circulating tumour cells in blood plasma. It also includes samples taken from a body fluid such as serum, plasma, blood, lymph, synovial, pleural, peritoneal, or cerebrospinal fluid, mucus, bile, urine saliva, tears and sweat. It will be appreciated that the methods of the invention encompasses taking a sample and quantitatively measuring the first biomarker from one of the aforementioned biological sources and making the first qualitative determination and that the quantitave determination of the second biomarker can be made from either the same or different biological source but always from the same individual and contemporaenously. Therefore, for example, and without limitation, the first biomarker quantitative value may be measured as cell numbers in a solid tumour whereas the second biomarker may be quantitatively measured as circulating levels in blood plasma. Accordingly, the quantitative measurement of the first biomarker maybe determined by a different quantitative method from the second biomarker. Thus the quantitative value of the first biomarker can be determined by cell number whereas the second biomarker can be quantitatively evaluated by circulating blood plasma levels. All such permutations are encompassed within the spirit of the present invention.

Reference herein to “contemporaneous” means that the sample for the first and second biomarkers may be taken simultaneously or within a specific time period such as minutes, hours, days or weeks. It will be understood that the essence of the invention k that the first and second biomarkers provide improved predictive values over each biomarker on its own and therefore the quantitative values for each biomarker should be assessed within a time period wherein the levels will be at at an almost identical time point.

Reference herein to a “biomarker” or “marker” are interchangeable and refers to a distinctive biological or biologically derived indicator of a process, event or condition. Predictive biomarker refers to a biomarker that can be used in advance or retrospectively of intervention/therapy to estimate response and/or survival of a patient on a specific treatment.

Reference herein to “selected pre-determined threshold value” refers to a cut off value below which the risk to a subject of a tumour having metastatic/invasive potential is minimal. In contrast, it is indicative of metastatic/invasive potential if either of both of the first and second biomarkers quantitative values are above or higher than the threshold value,

Reference herein to a “reference value” is the quantitative value obtained from individuals having a metastatic/invasive cancer. The reference value can be a numerical value for positively stained cells in a histopathological assessment of either the first and/or second biomarker or it can be the level of expression of either the first and/or second biomarker levels in a body fluid.

Reference herein to “predicting” refers to the act of anticipating a status or event and refers to making a finding that has an individual has a significantly enhanced or reduced probability of having a given status or experienced an event.

Preferably, the selected predetermined threshold value of the first biomarker is at least between 0.5 and 5.0%, for example, 1.0% or any other integer there between, of the total number of tumour cells in the sample. More preferably, the integer is at least 1.0% of a sample reference value.

Preferably, the threshold numbers for the second marker is at least biomarker is at least between 1-10.0%, for example, and preferably at least 5.0% of the total number of a sample reference value.

In one embodiment the sample reference value is the number of cells expressing the first and second biomarkers in subject known to have an invasive and/or metastatic tumour.

Preferably, the number of cells is assessed immunohisto/immunocyto-chemically and counted by manual cell counting, automated cell counting and/or indirect cell counting. For example, and without limitation the method includes manual cell counting (counting numbers in chambers; counting plating and CFU counting); automated cell counting (electrical resistance, flow cytometry, image analysis and stereological cell counting) and; indirect cell counting (spetrophtometry or impedence microbiology) hi some embodiments of the invention the sample reference value is the level of expression of the either first and/or second biomarkers in a subject having an invasive and/or metastatic tumour.

Preferably, wherein level of expression of the first and second biomarkers is assessed it is by ELISA, immunoprecipitation or immunoblotting.

Preferably, when the quantitative value of the first marker in the sample is lower than the selected predetermined threshold value for the first marker and the quantitative value of the second marker in the sample is lower than the selected predetermined threshold value for the second marker it indicates that the tumour does not have invasive and/or metastatic potential.

Preferably, when the quantitative value of the first marker in the sample is higher than the selected predetermined threshold value for the first marker and the quantitative value of the second marker in the sample is higher than the selected predetermined threshold value for the second marker indicates that the tumour does have invasive and/or metastatic potential.

Preferably, when the quantitative value of the first marker in the sample is lower than the selected predetermined threshold value for the first marker and the quantitative value of the second marker in the higher than the selected predetermined threshold value for the second marker it indicates that the tumour may have invasive and/or metastatic potential and requires further monitoring.

Preferably, when the quantitative value of the first marker in the sample is higher than the selected predetermined threshold value for the first marker and the quantitative value of the second marker in the lower than the selected predetermined threshold value for the second marker it indicates that the tumour may have invasive and/or metastatic potential and requires further monitoring.

Preferably the tumour is selected from the group of tumours comprising human breast cancer, and, in particular, an oestrogen receptor positive and human epidermal growth factor receptor 2 negative breast cancer cell or a triple receptor negative breast cancer (TRNBC) cell.

Preferably, the tumour status includes following initial diagnosis, monitoring for metastasis following surgery and/or during chemotherapy or radiotherapy, stratification of a group of subjects with cancer and/or predicting probability of survival.

Initial diagnosis of a tumor includes determining if a tumor is cancerous at all, whether it is a benign or non-cancerous growth or whether the tumor is malignant.

In another aspect of the invention, there is provided a kit comprising a first reagent and a second reagent for assessing respectively levels of a first marker and a second marker in a sample. The kit can also include instructions on how to perform the assay of the present invention.

The kit may be used in a method for determining whether a tumour in a subject has invasive and/or metastatic potential.

Preferably there is provided use of a kit as hereinbefore described for determining whether a tumour in a subject has invasive and/or metastatic potential.

In some embodiments, the method may determine number of tumour cells expressing the first marker and the second marker by immunohistochemical (IHC) staining of the sample.

The selected threshold number of tumour cells expressing a marker is a percentage number of tumour cells below which the sample is deemed not to significantly express the marker (a negative result) and at or above which the sample is deemed to significantly express the marker (a positive result).

As used herein, the expression “indicate that the tumour cell does not have invasive or metastatic potential” means that the probability the tumor cell is not invasive and/or metastatic (or is not progressing towards being invasive and/or metastatic) is equal to or greater than 90%, for example, 95% and preferably 96% or more, for example, 97%, 98%, 99% or 100%.

In one embodiment the invention provides a method for determining whether a sample obtained from a subject has invasive and/or metastatic in a whole tumour sample by staining of the first and scoring markers by comparison to a predetermined threshold level of cell numbers in a biopsy and a cut-off number of stained tumour cells.

As used herein, the term “tumour” is intended to be interchangeable with the term “cancer” and refers to multicellular tumours as well as individual neoplastic or pre-neoplastic cells and to refers to both primary and metastasized solid tumors and carcinomas of any tissue in a subject, including but not limited to breast; colon; rectum; lung; oropharynx; hypopharynx; esophagus; stomach; pancreas; liver; gallbladder; bile ducts; small intestine; urinary tract including kidney, bladder, and urothelium; female genital tract including cervix, uterus, ovaries (e.g., choriocarcinoma and gestational trophoblastic disease); male genital tract including prostate, seminal vesicles, testes and germ cell tumors; endocrine glands including thyroid, adrenal, and pituitary; skin (e.g., hemangiomas and melanomas), bone or soft tissues; blood vessels (e.g., Kaposi's sarcoma); brain, nerves, eyes, and meninges (e.g., astrocytomas, gliomas, glioblastomas, retinoblastomas, neuromas, neuroblastomas, Schwannomas and meningiomas).

In a further embodiment, the present invention provides a method for determining the probability of survival of a subject.

In a further embodiment, the method provides for stratification of a group of subjects to identify a sub-group of subjects when the determined number of tumour cells expressing the first marker is above or lower than the threshold number of cells expressing the first marker and the determined number of tumour cells expressing the second marker is lower than the threshold number of tumour cells expressing the second marker indicates that the tumour does not have invasive and/or metastatic potential.

The patients may be distinguished by those that ought to undergo chemotherapy or radiotherapy following surgery to remove the tumour and those that need not.

It will be appreciated that the methods of the invention are carried out in vitro, ex vivo or in silica. The sample may be tissue or cell material which is taken from a human or animal subject. In some embodiments, the method may determine the levels of the first marker and the second marker from a single tissue sample and, in particular, one or more portions of the tissue sample.

In some embodiments, the tumor is a primary and malignant cancer. In preferred embodiments, the tumour is a primary cancer derived from a human breast, ovarian, stomach, lung, brain, neck, pancreatic or colon cancer. In a particularly preferred embodiment, the tumour is a primary cancer derived from an ER +ve/HER2 −ve breast cancer or a triple receptor negative breast cancer (TRNBC).

Suitable reagents, antibodies and protocols for determining the expression of each marker in the sample will be known to the skilled person.

In some embodiments, the expression of the markers in the sample are determined by detecting a transcribed polynucleotide (or portion thereof) which encodes Ran or Ran binding protein 1 and detecting a transcribed polynucleotide (or portion thereof) which encodes for MMP2. The transcribed polynucleotides may be mRNA or cDNA. The transcribed polynucleotides may be amplified, for example, using a polymerase chain reaction (PCR or RT-PCR) prior to the determination. In these embodiments, the presence of each marker is determined by detecting respective polynucleotides which can bind to the transcribed polynucleotides (or portions thereof) under stringent hybridisation conditions.

In other embodiments, the expression of the markers are determined by detecting Ran or Ran binding protein 1 (or a fragment thereof) and detecting MMP2 (or a fragment thereof). The determination of each marker may use a respective reagent, such as an antibody, an antibody derivative or an antibody fragment, which specifically binds to one or other of these proteins. The determination may, in particular, use respective antibodies which are labelled, for example, by a radiolabel, a fluorophore label or an enzyme label. It may use respective antibody derivatives comprising an antibody conjugated to a substrate or a ligand or respective antibody fragments comprising a single chain antibody or an isolated antibody hypervariable domain.

In a further embodiment of the invention, the present invention provides a method for determining the probability of survival of a subject with a cancer by determining a quantitative value of the first and second biomarkers within a tumour cell obtained from a subject and comparing this to a selected pre-determined level.

When the intracellular levels of each marker are determined within a single tumour cell, the determined level of the first marker may be at least four, five, six, seven, eight or nine times lower than the reference/normal level for the first marker. Alternatively, or additionally, the determined level of the second marker may be at least four, five, six, seven, eight or nine times lower than the reference level of the second marker.

The levels of each marker in the subject and reference tumour cell can, for example, be determined using standard colorimetric methods. The levels of markers in the subject tumour cell can also be determined by comparison with respective absolute amounts or concentrations of each marker in a standard reference sample.

In some embodiments, the levels of the markers are determined by detecting a transcribed polynucleotide (or portion thereof) which encodes Ran or Ran binding protein 1 and detecting a transcribed polynucleotide (or portion thereof) which encodes for MMP2. The transcribed polynucleotides may be mRNA or cDNA. The transcribed polynucleotides may be amplified, for example, using a polymerase chain reaction (PCR or RT-PCR) prior to the determination.

In these embodiments, the level of each marker is determined by detecting the binding of respective reference polynucleotides which can bind to the transcribed polynucleotides (or portions thereof) under stringent hybridisation conditions.

The reference polynucleotides may be bound to a solid substrate or be labelled, for example, by a chromophore, a fluorophore, an enzyme, or an enzyme co-factor whereby to allow for determination of the subject polynucleotides by hybridisation. Alternatively, PCR or other techniques, for example, employing respective single nucleotide polymorphisms, can be used to determine the level of each marker.

In other embodiments, the levels of the markers are determined by detecting Ran or Ran binding protein 1 (or an active fragment thereof) and detecting MMP2 (or an active fragment thereof). The determination of each marker may use a respective reagent, such as an antibody, an antibody derivative or an antibody fragment, which specifically binds to one or other of these proteins. The determination may, in particular, use respective antibodies which are labelled, for example, by a radiolabel, a fluorophore label or an enzyme label. It may use respective antibody derivatives comprising an antibody conjugated to a substrate or a ligand or respective antibody fragments comprising a single chain antibody or an isolated antibody hypervariable domain.

In one embodiment of the invention there is provided a method for monitoring a tumour in a subject for invasive and/or metastatic potential. A therapy, for example, a drug treatment, surgical intervention or the like may be provided to the patient between the point in time when the levels of the first and second marker are initially determined and the later time point and the method will provide an indication as to whether the therapy is having an effect on the invasive and/or metastatic potential of the tumour.

The method may allow for the evaluation of different test agents and their ability to inhibit or bring about invasion and/or metastasis.

The method may comprise comparing the determined level of each marker in each of the aliquots wherein a significantly higher level of both markers in the aliquot exposed to the test agent as compared to the aliquot not exposed to the test agent is indicative that the test agent causes invasion and/or metastasis. In any case, the method may further comprise exposing further aliquots of the sample to further respective test agents.

The method may also provide a method for selecting an agent to inhibit invasion and/or metastasis of a tumour cell in a sample, the method comprising the steps of:
(i) providing at least first and second aliquots of the sample, wherein each aliquot comprises at least one tumour cell,
(ii) exposing the first aliquot of the sample to a first test agent,
(iii) exposing the second aliquot of the sample to a second test agent; and
(iv) determining the levels of a first marker and a second marker from the tumour cell in each aliquot;
wherein the first marker is selected from the group consisting of Ran, Ran binding protein 1, an active fragment of a Ran, a nucleic acid sequence encoding Ran, a nucleic acid sequence encoding Ran binding protein 1, a nucleic acid sequence encoding an active fragment of Ran and a nucleic acid sequence encoding an active fragment of Ran binding protein 1; and
the second marker is selected from the group consisting of MMP2, an active fragment of MMP2, a nucleic acid sequence encoding MMP2 and a nucleic acid sequence encoding an active fragment of MMP2; and
(v) comparing the determined levels of each marker in each of the aliquots; and
(vi) selecting the test agent which provides for a lower determined level of at least the second marker in an aliquot with that test agent as compared to an aliquot with another test agent.

The method may comprise selecting the test agent which provides for a lower determined level in at least the second marker in an aliquot with that test agent as compared to an aliquot with the other test agent. In any case, the method may further comprise exposing further aliquots of the sample to further respective test agents.

The selection of a test agent which provides lower levels of the second marker or of both markers in an aliquot with that agent as compared to other aliquots with other test agents may be utilised to provide a medicament to the patient.

The method may also find utility in identifying an agent that inhibits invasion or metastasis of a tumour in a subject comprising the steps of:
(i) taking a sample from a tumour of the subject;
(ii) providing at least first and second aliquots from the sample, wherein each aliquot comprises at least one tumour cell,
(ii) exposing the first aliquot to a first test agent,
(iii) exposing the second aliquot to a second test agent,
(iv) determining the levels of a first marker and a second marker from the tumour cell of each aliquot,
wherein the first marker is selected from the group consisting of Ran, Ran binding protein 1, an active fragment of a Ran, a nucleic acid sequence encoding Ran, a nucleic acid sequence encoding Ran binding protein 1, a nucleic acid sequence encoding an active fragment of Ran and a nucleic acid sequence encoding an active fragment of Ran binding protein 1; and
the second marker is selected from the group consisting of MMP2, an active fragment of MMP2, a nucleic acid sequence encoding MMP2 and a nucleic acid sequence encoding an active fragment of MMP2; and
(v) comparing the determined levels of each marker in each of the aliquots and (vi) selecting the test agent which provides for a lower level of at least the second marker in an aliquot with that test agent as compared to an aliquot with another test agent; and
(vi) administering to the patient a therapeutically effective amount of the selected test agent.

The method may comprise selecting the test agent which provides for a lower determined level in each marker in an aliquot with that test agent as compared to an aliquot with the other test agent. In any case, the method may further comprise exposing further aliquots of the sample to further respective test agents.

In one aspect of the invention there is provided a medicament comprising therapeutic amounts of a Ran inhibitor and a MMP2 inhibitor.

In a further aspect, the present invention also provides kits for carrying out the foregoing methods. The present invention may provide a kit for determining tumor status in a subject, the kit comprising a first reagent and a second reagent for assessing respectively levels of a first marker and a second marker in a tumour sample.

A kit may also comprise suitable visual indicators for the reagents, for example, labelled antibodies for each reagent capable of binding to the reagent and/or the reagent and marker complex. A kit may optionally comprise liquids such as buffers suitable for detecting the level of the first and second markers in a sample, for example, buffers which provide for binding an antibody specific to Ran and/or MMP2.

In embodiments, the antibody, antibody derivative or antibody fragment used to measure the level of the first and second markers may be labelled with a radiolabel, a flurophore label or an enzyme label. Antibody derivatives can comprise antibodies or antibody fragments which are conjugated with a substrate or ligand. An antibody fragment can be, for example, a single chain antibody or an isolated antibody hypervariable domain.

Note, having regard to the foregoing methods, that the description of any feature in respect of one method is also a description of in respect of the other methods unless the context demands otherwise. Each any every feature ascribed to one embodiment of aspect of the invention applies mutatis mutandis to each any every other aspect of the invention.

The present invention is now described in more detail with reference to the following Examples and the accompanying drawings in which:

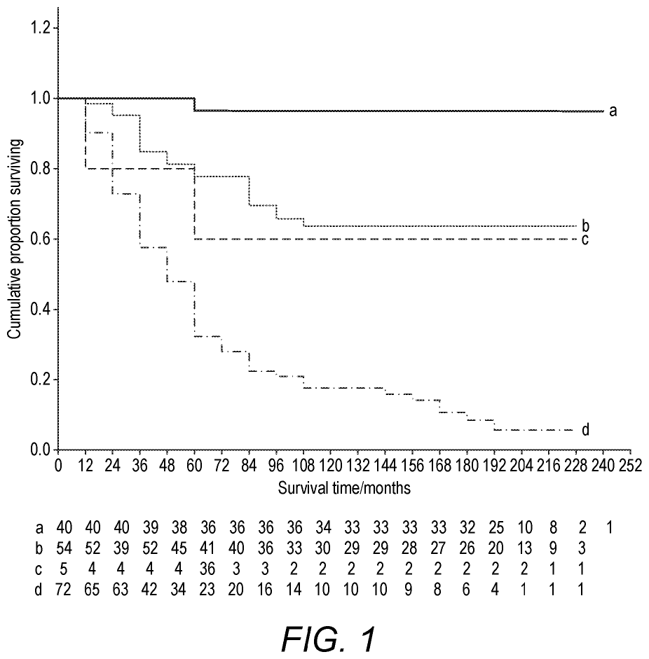

FIG. 1 is graph plotting the cumulative proportion of surviving patients against survival time as determined by a retrospective immunohistochemical study of sections of primary tumours taken from the 181 unselected human breast cancer patients—classified (a to d) according to positive and/or negative staining for Ran protein and for MMP2 protein;

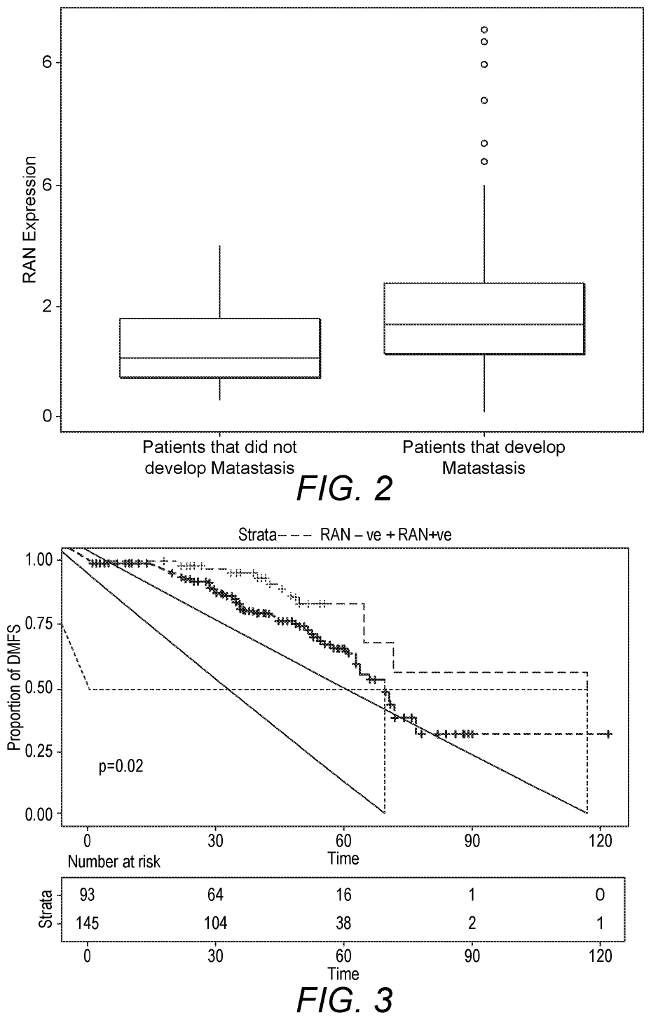

FIG. 2 is a graph showing the extent of RAN expression in the blood plasma of 238 unselected breast cancer patients which went on to develop metastasis as compared to patients that did not go onto develop metastasis; and

FIG. 3 is a graph plotting the proportion of distance metastasis free survival (DMFS) against time as determined by retrospective Elisa assay of RAN levels (positive and negative) in the blood plasmas of the 238 cancer patients.

### EXAMPLE 1

A retrospective statistical study was undertaken using (−/+) IHC staining of samples of 181 primary tumours from unselected breast cancer patients in order to determine the relationship between Ran, c-Met, c-Myc and MMP2.

The study was conducted in accordance with methods previously described for Ran (de Silva Rudland S. et al in in Am. J. Pathol. 2011, 79, 1061-1072 and Rudland P. S. et al in Am. J. Pathol. 2010, 176, 2935-2947). Briefly, patients received no adjuvant therapy including hormonal therapy and only patients with operable breast cancer (T1-4, NO-1) were included. Patient follow-up times ranged from 14.5 to 19.4 years (mean 16.4±0.1 years) with a mean±SE survival time of 9.0±0.5 years. Ethical approval was obtained from NRES Committee North West REC Ref 12/NW/0778, Protocol no. UoL000889, IRAS no 107845. Samples were preserved in neutral buffered formalin and embedded in paraffin wax as described previously (Rudland P. S. et al in Cancer Res. 2000, 60, 1595-1603).

### Materials and Methods

IHC staining. Histological sections cut at 4 μm were mounted on slides, treated with 0.05% v/v H₂O₂ in methanol to inhibit endogenous peroxidase (Rudland P. S. et al, in Cancer Res. 2000, 60, 1595-1603) and incubated with the relevant primary and horseradish peroxidase labelled antibodies/polymers in kits (DAB) (Dako Ltd, Ely, UK), as described previously (de Silva Rudland S. et al, in Am. J. Pathol. 2011, 79, 1061-1072 and Ismail T. M. et al, in Cancer Res. 2017, 77, 780-789). Positive staining corresponded to an oxidised brown precipitate of diaminobenzidine (DAB). Slides were finally mounted in Glycergel mounting medium (Dako). Blocked antibodies prepared by mixing 1 mg/ml of the relevant blocking peptide/protein abolished this staining. Appropriate immune serum also yielded no staining. Western blots of breast cell lines verified the specifity of all antibodies used by yielding the appropriately-sized molecular weight bands on SDS—polyacrylamide gels.

IHC scoring analysis. IHC-stained sections were analysed and scored by two independent observers using light microscopy according to the percentage of stained carcinoma cells from 2 well separated sections of each specimen, 10 fields per section at 200× magnification and a minimum of 200 cells per field, as described previously (de Silva Rudland S. et al, in Am. J. Pathol. 2011, 79, 1061-1072 and Ismail T. M. et al, in Cancer Res. 2017, 77, 780-789).

Staining data analysis was performed using Excel (Microsoft, Redmond, Wash.), and SPSS version 22 (SPSS, Chicago, Ill.).

Staining for all proteins had already been separated into two categorical groups, a negative and positive group with a cut-off of either 1% or 5% of carcinoma cells staining, according to which cut-off yielded the more significant difference and greater relative risks: 1% cut-offs for Ran, cMyc, Ki67, CK5/6 and 5% cut-offs for cMet, MMP2, ERα, c-erbB-2, PgR (de Silva Rudland S. et al in Am. J. Pathol. 2011, 79, 1061-1072; Yuen H-F. et al in Clin. Cancer Res. 2012, 18, 380-391; Yuen H-F. et al in J. Natl. Cancer Inst. 2013, 105, 475-488; Yuen H-F. et al in Oncotarget 2016, 7, 75854-75864; and Ismail T. M. et al, in Cancer Res. 2017, 77, 780-789).

The association of staining for each protein separately in this set of patients was calculated from life tables constructed from survival data using Kaplan Meier plots and analysed by Wilcoxon (Gehan) statistics (Rudland P. S. et al in Cancer Res. 2000, 60, 1595-1603). Patients who died from causes other than cancer were censored. Unadjusted relative risk (RR) for survival with 95% confidence interval (95% CI) was calculated using Cox's univariate analysis (Rudland P. S. et al in Am. J. Pathol. 2010, 176, 2935-2947).

Association of IHC staining for Ran or MMP2 with other tumour variables was assessed by cross-tabulations using Fishers Exact test (2-sided) using either 1% or 5% cut-offs. For multiple comparisons the resultant P values were corrected by the Holm-Bonferroni formulae of I—(I-P)ⁿ, where n is the number of tumour variables.

Binary Logistic Regression was used for calculation of the relative independent association (RA) of staining for one protein with the remaining proteins in the group. To determine if the association of patient survival with Ran, MMP2 etc. was significant within a group of proteins, Cox's multivariate analyses were performed on 181 patients, incomplete data arose mainly from lack of sampling (de Silva Rudland S. et al, in Am. J. Pathol. 2011, 79, 1061-1072).

Association of Individual Tumour Variables with Patient Survival Times

The relationship in human breast cancer of tumour variables including markers Ran, c-Met, c-Myc, MMP2, Ki67, Era, c-erbB-2 and CK5/6 with patient demise as a result of metastatic breast cancer was investigated as described above.

The results of the statistical analyses are shown in Table 1. As may be seen, the largest significant differences in relative risk (RR) within the present group of 181 patients were as follows: Ran (χ²=35.4, RR=14.9), cMet (χ²=32.9, RR=10.7), cMyc (χ²=40.3, RR=9.5), MMP-2 (χ²=64.8, RR=7.7) and CK5/6 (χ²=43.3, RR=5.6).

The differences in relative risk for ERα (χ²=1.24, RR=0.81), c-erbB-2 (χ²=1.93, RR=1.33) and Ki67 (χ²=1.6, RR=1.3) were not found to be significant within this group of patients—notwithstanding a report that in a larger patient group they significantly different (de Silva Rudland S. et al, in Am. J. Pathol. 2011, 79, 1061-1072).

In contrast to this report, the present group of patients showed a significant RR for nodal size (with or without the involvement of lymph nodes) of 2.3 (χ²=14.64, 1df, P<0.001). The tumour size and histological grade were not found to be significantly associated with patient survival.

Association of Ran and MMP2 with Other Tumour Variables

The association in human breast cancer of Ran with the other tumour variables and of MMP2 with other tumour variables was investigated as described above. The results are summarised in Table 2.

As may be seen, Ran was very significantly associated with c-Met (P=6.6×10⁻⁵), cMyc (P=4.4×10⁻⁵), MMP2 (P=5.7×10⁻⁶), and CK5/6 (P=5.5×10⁻⁵) but not at all associated with Ki67, ERα, c-erbB-2, tumour size and histological grade (P 0.34).

The association of Ran with TRNBC (P uncorrected=0.06) was of borderline significance and involved lymph nodes alone (P uncorrected=0.037). The most significant association of Ran with other tumour markers was found to be the association between Ran and MMP2.

MMP2 was strongly significantly associated with the same tumour markers as Ran viz., c-Met (P=6.4×10), c-Myc (P=1.8×10⁻⁷), CK5/6 (P=7.5×10⁻⁷) as well as with RAN itself (P=5.7×10⁻⁶) and not with the remainder of the tumour variables (P 0.35).

Note that there is no significant staining for Ran with staining for Ki67 which appears consistent with the fact that little increase in cell proliferation is observed in Ran transfected cells.

Further, the fact that staining for Ran, c-Met, c-Myc and MMP2 are all very significantly associated with staining for CK5/6 but not staining for Era or c-erbB-2 suggests that RAN c-Met, c-Myc and MMP2 occur mainly in the Basal Cell type of breast cancers.

This sub-group of breast cancers overlaps considerably with the triple receptor negative breast cancer (TRNBC) sub-group (de Silva Rudland S. et al, in Am. J. Pathol. 2011, 79, 1061-1072) which may explain the borderline association of Ran staining with triple receptor negative breast cancer.

TABLE 1
Association of tumour variables with patient survival times

| | | | | Cum. | | | | |
|---|---|---|---|---|---|---|---|---|
| | | | Median | survival | | | | |
| Tumour | Patient | | survival | at end | | | | |
| variable^{a} | no | Grouping | (months) | (%) | χ^{2 b} | P ^{b} | RR ^{c} | 95% CI ^{c} |
| Ran | 181 | − | 228 | 93 | 35.44 | <0.001 | 14.87 | 4.69-47.15 |
| | | + | 73.3 | 32 | | | | |
| Met ^{d} | 162 | − | 216 | 90 | 32.95 | <0.001 | 10.71 | 4.30-26.64 |
| | | + | 59.6 | 26 | | | | |
| cMyc | 181 | − | 228 | 88 | 40.31 | <0.001 | 9.49 | 4.13-21.80 |
| | | + | 58.9 | 29 | | | | |
| MMP2 ^{d} | 181 | − | 228 | 78 | 64.82 | <0.001 | 7.70 | 4.67-12.70 |
| | | + | 48.4 | 10 | | | | |
| Ki67 | 147 | − | 189.3 | 49 | 1.58 | 0.21 | 1.31 | 0.81-2.10 |
| | | + | 83.9 | 42 | | | | |
| ERα ^{d} | 179 | − | 103.9 | 46 | 1.24 | 0.265 | 0.81 | 0.53-1.25 |
| | | + | 228 | 51 | | | | |
| c-erbB-2 ^{d} | 176 | − | 185.0 | 49 | 1.93 | 0.165 | 1.33 | 0.82-2.15 |
| | | + | 59.8 | 44 | | | | |
| CK5/6 | 177 | − | 228 | 72 | 43.29 | <0.001 | 5.57 | 3.54-8.78 |
| | | + | 50.6 | 07 | | | | |
| TRNBC^{e} | 164 | − | 216 | 55 | 0.37 | 0.545 | 1.23 | 0.72-2.11 |
| | | + | 156.1 | 45 | | | | |
| Nodal | 136 | − | 228 | 58 | 14.64 | <0.001 | 2.32 | 1.43-3.77 |
| status ^{f} | | + | 59.1 | 35 | | | | |
| Grade ^{g} | 164 | − | 173.7 | 48 | 2.41 | 0.12 | 1.4 ^{g} | 0.88-2.24 ^{g} |
| | | (Gr1, 2) | | | | | | |
| | | + | 58.8 | 40 | | | | |
| | | (Gr3) | | | | | | |
| Tumour | 175 | − | 216 | 51 | 1.02 | 0.31 | 1.33 ^{h} | 0.82-2.16 ^{h} |
| size ^{h} | | (T₁, T₂) | | | | | | |
| | | + | 80.0 | 39 | | | | |
| | | (T₃, T₄) | | | | | | |

^{a}Negative vs positive staining, 1% cut-off, except where stated.
^{b} χ² and probability (P) were determined using generalised Wilcoxon (Gehan) statistics.
^{c} RR and 95% CI were determined using Cox's univariate analysis with 1 df.
^{d} Negative vs. positive staining, 5% cut-off.
^{e}Triple Receptor Negative Breast Cancer (TRNBC− vs TRNBC+).
^{f} No nodes vs. 1 or more nodes; P for 1 df.
^{g} Histological grade: 1, 2 vs 3; P for 1 df. RR for Grade 1 vs 2 = 2.74 (95% CI, 1.45-5.19), 1 vs 3 = 2.83 (1.42-5.66); all 1 df.
^{h} Tumour size <5 cm vs >5 cm in diameter, T₁, T₂ vs T₃, T₄; P for 1 df. RR for T₁, T₂ vs T₃, T4; T₁ vs T₂ = 1.54 (95% CI, 0.67-3.58), T₁ vs T₃ = 1.76 (0.69-5-4.51), T₁ vs T₄ = 2.68 (0.86-8.32), all 1 df.

TABLE 2
Association of IHC staining for Ran
and MMP2 with other tumour variables

| | | |
|---|---|---|
| Tumour | Patient | Statistical significance^{c} |

| variable^{a} | no^{b} | RAN | MMP2 |
|---|---|---|---|
| Ran | 181 | — | 5.7 × 10⁻⁶ |
| cMet^{d} | 162 | 6.6 × 10⁻⁵ | 6.4 × 10⁻⁹ |
| cMyc | 181 | 4.4 × 10⁻⁵ | 1.8 × 10⁻⁷ |
| MMP2^{d} | 181 | 5.7 × 10⁻⁶ | — |
| Ki67 | 147 | 0.94 | 0.50 |
| ERα^{d} | 179 | 0.94 | 1.0 |
| c-erbB-2^{d} | 176 | 1.0 | 0.85 |
| CK5/6 | 177 | 5.5 × 10⁻⁵ | 7.5 × 10⁻⁷ |
| TNRBC^{e} | 164 | 0.50^{f} | 0.97 |
| Tumour size^{g} | 175 | 0.99 | 0.83 |
| Grade^{h} | 164 | 0.99 | 1.0 |
| Nodeⁱ | 136 | 0.34^{j} | 0.35^{j} |

^{a}Negative vs positive IHC staining for molecular variables using 1% cut-off, except where stated.
^{b}Number of patients from original 181.
^{c}Probability P from Fisher's Exact test using the Holm-Bonferroni correction calculated as 1 − (1 − P)ⁿ, where n = 11.
^{d}Negative vs positive staining for 5% cut-off.
^{e}Triple Receptor Negative Breast Cancer.
^{f}Without Bonferroni correction P = 0.061.
^{g}Tumour size <5 cm vs >5 cm in diameter.
^{h}Histological grade 1, 2 vs 3.
ⁱNo nodes vs 1 or more lymph nodes involved.
^{j}Without Bonferroni correction P = 0.037 for Ran and 0.039 for MMP2.

Relative Independent Association of Ran, c-Met, c-Myc, MMP2 and Ki67

The probability of independent association in human breast cancer of Ran, c-Met, c-Myc and Ki67 with each other was investigated as described above.

The results are shown in Table 3. As may be seen, the relative independent association (RA) of Ran with cMet and with MMP2 was found to be strongest (RA=3.0 to 3.4), whilst the relative independent association of Ran with Ki67 was found not to be significant (RA=1.12, P=0.81).

TABLE 3
Probability of independent association of staining
for Ran and other molecular markers

| Test^{a} | Other^{b} | | | | | | |
|---|---|---|---|---|---|---|---|
| variable | variables | Coeff β^{c} | SE of β^{c} | χ^{2 d} | P^{e} | RA^{f} | 95% CI^{f} |
| Ran | cMet | 1.221 | 0.536 | 5.185 | 0.023 | 3.39 | 1.19-9.69 |
| | cMyc | 0.677 | 0.521 | 1.687 | 0.194 | 1.97 | 0.71-5.47 |
| | MMP2 | 1.099 | 0.628 | 3.064 | 0.080 | 3.00 | 0.88-10.27 |
| | Ki67 | 0.117 | 0.493 | 0.056 | 0.813 | 1.12 | 0.43-2.96 |
| cMet | Ran | 1.228 | 0.525 | 5.462 | 0.019 | 3.41 | 1.22-9.56 |
| | cMyc | 0.866 | 0.498 | 3.018 | 0.082 | 2.38 | 0.90-6.31 |
| | MMP2 | 2.067 | 0.566 | 13.327 | <0.001 | 7.9 | 2.60-23.96 |
| | Ki67 | 0.488 | 0.484 | 1.015 | 0.314 | 1.63 | 0.63-4.21 |
| cMyc | Ran | 0.697 | 0.515 | 1.833 | 0.176 | 2.01 | 0.73-5.50 |
| | cMet | 0.852 | 0.504 | 2.856 | 0.091 | 2.34 | 0.87-6.30 |
| | MMP2 | 1.644 | 0.550 | 8.923 | 0.003 | 5.18 | 1.76-15.22 |
| | Ki67 | 0.079 | 0.469 | 0.029 | 0.866 | 1.08 | 0.43-2.72 |
| MMP2 | Ran | 0.982 | 0.627 | 2.451 | 0.117 | 2.67 | 0.78-9.13 |
| | cMet | 2.047 | 0.571 | 12.869 | <0.001 | 7.74 | 2.53-23.70 |
| | cMyc | 1.587 | 0.553 | 8.238 | 0.004 | 4.89 | 1.65-14.46 |
| | Ki67 | 0.305 | 0.468 | 0.424 | 0.515 | 1.36 | 0.54-3.40 |

^{a}Principle IHC-staining variable for probability of association with other tumour variables using cut-offs defined in Tables 1, 2.
^{b}Sets of other IHC-staining variables were included in binary Logistic Regression Analysis using cut-offs defined in Tables 1, 2 to separate positive and negative staining groups.
^{c}Value of coefficient β (Coeff β) with its standard error (SE) in binary Logistic Regression Analysis.

c-Met showed the strongest relative independent associations with Ran (RA=3.41, P=0.019) and with MMP2 (7.9, P<0.001).

The strongest relative independent association for c-Myc was with MMP2 and the strongest relative independent association of MMP2 was with cMet.

Association of Ran, cMet, cMyc and MMP2 with Patient Survival

The stainings for Ran, cMet, cMyc and MMP2 were investigated for relative independent association with patient survival times as described above.

The results are shown in Table 4. As may be seen, the set of stainings for Ran, c-Met, c-Myc and MMP2 showed a significant degree of independence (P≤0.036) with similar relative risks (RR) for patient demise of 3.1 fold to 3.7 fold. These RRs are considerably less than the 7 fold to 15 fold decreases shown in univariate analyses reported in Table 1.

The sets of stainings for Ran and c-Met and Ran and MMP2 showed a reduction in RR for patient demise as compared to Ran staining alone (from 14.9 fold to between 7.6 to 7.8 fold). The set of stainings for Ran and c-Myc showed a reduction in RR for patient demise as compared to Ran staining alone (from 14.9 fold to 9.8 fold).

TABLE 4
Summary of results for Cox's proportional
hazards for cancer-related deaths

| Tumour | | | | | | |
|---|---|---|---|---|---|---|
| variable^{a} | Coeff β^{b} | SE of β^{b} | χ^{2c} | P^{d} | RR^{e} | 95% CI^{e} |
| Set A | | | | | | |
| Ran | 1.305 | 0.622 | 4.398 | 0.036 | 3.69 | 1.09-12.49 |
| cMet | 1.153 | 0.503 | 5.257 | 0.022 | 3.17 | 1.18-8.49 |
| cMyc | 1.246 | 0.445 | 7.827 | 0.005 | 3.48 | 1.45-8.32 |
| MMP2 | 1.127 | 0.310 | 13.225 | <0.001 | 3.10 | 1.68-5.67 |
| Set B | | | | | | |
| Ran | 2.025 | 0.594 | 11.600 | 0.001 | 7.57 | 2.36-24.28 |
| cMet | 1.985 | 0.470 | 17.879 | <0.001 | 7.28 | 2.90-18.27 |
| Set C | | | | | | |
| Ran | 2.286 | 0.592 | 14.918 | <0.001 | 9.84 | 3.08-31.39 |
| cMyc | 1.873 | 0.427 | 19.252 | <0.001 | 6.51 | 2.82-15.02 |
| Set D | | | | | | |
| Ran | 2.056 | 0.600 | 11.727 | 0.001 | 7.82 | 2.41-25.36 |
| MMP2 | 1.642 | 0.260 | 40.023 | <0.001 | 5.17 | 3.11-8.59 |

^{a}In Set A comparisons were made between duration of survival time of patients with tumours stained for Ran, cMet, cMyc and MMP2; overall χ² = 96.21, 4 df, P < 0.001. In Set B comparisons between patients with tumours stained for Ran and cMet; overall χ² = 52.4, 2 df, P < 0.001. In Set C comparisons between patients with tumours stained for Ran and cMyc; overall χ² = 60.6, 2 df, P < 0.001. In Set D comparisons between patients with tumours stained for Ran and MMP2; overall χ² = 96.5, 2 df, P < 0.001. IHC cut-offs as described in Tables 1, 2.
^{b}Value of β coefficient (=logₑRR) and standard error (SE) in Cox's multiple regression analysis.
^{c}Cox's statistic χ².
^{d}Probability P from Cox's statistic χ², 1 df in each case.
^{e}Relative Risk (RR) for survival and 95% confidence interval (95% CI) from multivariate analysis.

Note that the stronger relative independent association between staining for MMP2, c-Met and c-Myc than staining for Ran suggests that the increase in MMP2 expression in tumours is not solely due to an increase in Ran but may arise from other signalling mechanisms.

Further, the partial nature of the confounding of RR for Ran suggests that other pathways not involving Ran are also involved in causing patient demise.

Note that the decline in RR for Ran staining with either c-Met staining or MMP2 staining is binary combination with Ran (49% and 48% respectively) was larger than the decline in RR for staining for Met or MMP2 staining in binary combination with Ran (32% for both) suggests that Met and MMP2 are more proximal members in this signalling pathway than Ran.

Association of Ran and MMP2 with Patient Survival Times

Table 5 and FIG. 1 shows cumulative proportions of surviving patients as a fraction of the total for each year (up to about 20 years) after presentation with carcinomas classified according to the following sets:

Set a (solid line): negatively stained for Ran (−ve) and for MMP2 (−ve); set b (dotted line): positively stained for Ran (+ve) and negatively stained for MMP2 (−ve) set c (dashed line): negatively stained for Ran (−ve) and positively stained for MMP2 (+ve); and set d (dashed and dotted line): positively stained for both Ran (+ve) and MMP2 (+ve).

As may be seen, in set a the median survival (ms) was greater than 228 months, the final cumulative survival (fcs) was 0.97. There were 39 censored observations (8 dead of other causes); in set b the ms was greater than 216 months and the fcs was 0.6. There were 44 censored observations (19 dead of other causes). In set c the ms was greater than 216 months and the fcs was 0.60. There were 3 censored observations (1 dead of other causes). In set d, the ms was 46.2 months and the fcs was 0.06. There were 8 censored observations (4 dead of other causes).

The statistical analysis of staining groups consisting of two of these sets shows that staining for Ran and for MMP2 can be synergistic and increase RR respectively from 17.1 fold and 23.1 fold to 82.1 fold (compare staining set a against staining set d). In terms of patient survival, the staining may show a reduction in patient survival after nearly 20 years respectively from 64% and 60% to only 6%.

TABLE 5
Difference in survival between staining sets

| | | | | | | Median | |
|---|---|---|---|---|---|---|---|
| Staining | FIG. 1 | | | | | survival ^{d} | Cumulative |
| Group^{a} | line | χ^{2 b} | P ^{b} | RR ^{c} | 95% CI ^{c} | (months) | survival ^{d} |
| Ran−/MMP2− | a vs b | 14.02 | <0.001 | 17.11 | 2.30-127.56 | >228 | 0.97 |
| Ran+/MMP2− | | | | | | >216 | 0.64 |
| Ran−/MMP2− | a vs c | 10.583 | 0.001 | 23.10 | 2.09-255.02 | >228 | 0.97 |
| Ran−/MMP2+ | | | | | | >216 | 0.60 |
| Ran+/MMP2− | b vs c | 0.407 | 0.524 | 1.35 | 0.32-5.78 | >216 | 0.64 |
| Ran−/MMP2+ | | | | | | >216 | 0.60 |
| Ran+/MMP2− | b vs d | 33.09 | <0.001 | 4.80 | 2.88-7.99 | >216 | 0.64 |
| Ran+/MMP2+ | | | | | | 46.2 | 0.06 |
| Ran−/MMP2+ | c vs d | 1.569 | 0.210 | 3.56 | 0.87-14.61 | >216 | 0.60 |
| Ran+/MMP2+ | | | | | | 46.2 | 0.06 |
| Ran−/MMP2− | a vs d | 59.643 | <0.001 | 82.11 | 11.34-594.64 | >228 | 0.97 |
| Ran+/MMP2+ | | | | | | 46.2 | 0.06 |

^{a}Immunocytochemical staining class for MMP2 and Ran classified as staining (+; +ve) or not staining (−; −ve).
^{b} Wilcoxon statistic (χ²) and Probability (P) were determined using the generalised Wilcoxon (Gehan) test with 1 df..
^{c} Relative risk (RR) and confidence interval (95% CI) were determined using Cox's univariate analysis with 1 df.
^{d} Median survival in months and cumulative proportion of patients surviving evaluated using life tables constructed from survival data.

A significant decrease in patient survival times is found for the doubly stained set d (Ran +ve/MMP2 +ve) over the singly-stained set b (Ran +ve/MMP2 −ve; P<0.001) which is not found for the doubly stained set d (Ran +ve/MMP2 +ve) over the singly stained set b (Ran −ve/MMP2 +ve; P=0.21).

This is particularly clear when cumulative proportion of surviving patients is plotted against survival time for each staining group. As may be seen from FIG. 1, noting that the number of patients presenting each year is set out below that figure, the curves corresponding to each staining group are highly significantly different from one another.

Note that Table 5 shows that when staining data for MMP2 is added to staining data for Ran in primary cancer cells, there is a significant decrease in patient survival times—but that when staining data for Ran is added to staining data for MMP2 there is no significant increase in patient survival times. So much supports the notion that the c-Met and MMP2 are more proximal members than Ran in the pathway leading to patient demise and is consistent with the order of the proteins that leads to an increase in metastatic properties of cultured cells.

TABLE 6
Sensitivity, Specificity, NPR and PPR in IHC assay for Ran and MIVIP2 alone and in combination

| | | | | | Patients | | | | | | | |
|---|---|---|---|---|---|---|---|---|---|---|---|---|
| | | IHC | Patients | | died from | | Total | | | | | |
| | | Staining | Alive | | cancer | | Patients | | Sensitivity | Specificity | | |
| | IHC | Score at | | % | | % | | % | (true +ve | (true −ve | | |
| Sub-Type | Assay | Diagnosis | # | alive | # | died | # | total | rate) | rate) | PPR | NPR |
| All | Ran | <1% | 42 | 44.7 | 3 | 3.4 | 45 | 24.9 | | 44.7 | | 93.3 |
| | | (−ve) | | | | | | | | | | |
| All | Ran | 2-5% | 52 | 55.3 | 84 | 96.6 | 113 | 75.1 | 96.6 | | 74.3 | |
| | | (+ve) | | | | | | | | | | |
| All | Ran | All | 94 | 100 | 87 | 100 | 181 | 100 | | | | |
| ER+/HER− | Ran | <1% | 23 | 51.1 | 1 | 1.9 | 24 | 24.7 | | 51.1 | | 95.8 |
| | | (−ve) | | | | | | | | | | |
| ER+/HER− | Ran | 2-5% | 22 | 48.9 | 51 | 98.1 | 73 | 75.3 | 98.1 | | 69.7 | |
| | | (+ve) | | | | | | | | | | |
| ER+/HER− | Ran | All | 45 | 100 | 52 | 100 | 97 | 100 | | | | |
| Triple −VE | Ran | <1% | 28 | 84.8 | 2 | 7.1 | 30 | 49.2 | | 84.8 | | 93.3 |
| | | (−ve) | | | | | | | | | | |
| Triple −VE | Ran | 2-5% | 5 | 15.2 | 26 | 92.9 | 31 | 50.8 | 92.9 | | 84.2 | |
| | | (+ve) | | | | | | | | | | |
| Triple −VE | Ran | All | 33 | 100 | 28 | 100 | 61 | 100 | | | | |
| All | MMP2 | <2% | 83 | 88.3 | 21 | 24.1 | 104 | 57.5 | | 88.3 | | 79.8 |
| | | (−ve) | | | | | | | | | | |
| All | MMP2 | 2-5% | 11 | 11.7 | 66 | 75.9 | 77 | 42.3 | 75.9 | | 91.7 | |
| | | (+ve) | | | | | | | | | | |
| All | MMP2 | All | 94 | 100 | 87 | 100 | 181 | 100 | | | | |
| All | Ran/MMP2 | −ve/−ve | 39 | 83.0 | 1 | 1.5 | 40 | 32.0 | | 83.0 | | 97.5 |
| All | Ran/MMP2 | +ve/+ve | 8 | 17 | 64 | 98.5 | 72 | 68.0 | 98.5 | | 88.8 | |
| All | Ran/MMP2 | All | 47 | 100 | 65 | 100 | 112 | 100 | | | | |

The present study shows that the addition of staining data for MMP2 to staining data for Ran expression reveals a more accurate picture of the metastatic potential of human breast cancer cells.

In this study, when staining data for MMP2 expression is added to that for Ran expression, the RR for patient death is increased from the original 14.9 to 82.1 fold.

It will be seen that Example 1 provides an immunohistochemical assay based on Ran and MMP2 which is better correlated with patient survival as compared to immunohistochemical assay based on Ran alone (compare NPR of 97.5% with 93.3%). The improvement in NPR strongly suggests a corresponding improvement in assay of Ran and MMP2 levels within a tumour cell and blood plasma as compared to assay of Ran alone.

Table 6 above summarises the present inventors knowledge of the sensitivity, specificity, the negative percent response (NPR) and the positive percent response (PPR) for Ran expression and MMP2 expression in IHC assay as compared to Ran alone in selected human breast cancer sub-types (ER +ve/HER −ve (ER +/HER−) and triple negative (triple −VE)) as well as in unselected (all) human breast cancers.

### EXAMPLE 2

A retrospective cohort study was undertaken of Ran levels in the blood plasma of 238 unselected human breast cancer patients (9236 cohort). The study utilised an Enzyme-Linked Immunosorbent Assay (ELISA) assay to determine the levels of Ran in blood plasma samples taken when the patients were first diagnosed with breast cancer.

Table 7 shows the mean Ran expression in patients that developed metastasis as compared to patients that did not not develop metastasis.

TABLE 7
Mean Ran expression with/without metastasis

| Patients | Number | Mean Ran expression |
|---|---|---|
| Patients that did not go on to | 177 | 1.33 |
| develop metastasis | | |
| Patients that went on to | 61 | 2.08 |
| develop metastasis | | |

A statistical analysis was made on the determined levels of Ran against patient survival to determine a cut-off level for Ran which in combination with assay for MMP2 is considered likely to give a clinically useful test with NPR of about 98%.

FIG. 2 plots the variation in Ran expression in patients that went on to develop metastasis as compared to patients that did not develop metastasis in this cohort.

The blood plasma Ran levels were analysed at a series of cut-off levels at which levels of Ran in the blood plasma above a percentage integer were considered positive (+ve) viz. to indicate that a patient would go on to develop metastasis.

Table 8 shows a sensitivity analysis for Ran expression at different half-integer cut-off levels between 0.5 and 2.0.

As may be seen, the number of patients who are Ran +ve when the cut-off level is 0.5 is very high (at 216) having regard to the number of true positives (61, shown in Table 10). Furthermore, the sensitivity and NPR is high and the specificity acceptable but the number of patients who are Ran −ve is very low (at 30% of total true negative).

TABLE 8
Sensitivity analysis at different Ran expression cut-offs

| Ran expression | Split | % | % | | |
|---|---|---|---|---|---|
| cut-offs | +ve/−ve | sensitivity | specificity | PPV | NPV |
| 0.5 | 216/22 | 96.7 | 11.3 | 27.3 | 90.9 |
| 1 | 145/93 | 80.3 | 45.8 | 33.8 | 87.1 |
| 1.5 | 95/143 | 55.7 | 65.5 | 35.8 | 81.1 |
| 2 | 63/175 | 37.7 | 77.4 | 36.5 | 78.3 |

Although the cut-off level of 0.5 appears to offer a relatively safe test in that of the 22 patients who would not have undertaken chemotherapy, 90% (20) would not have developed metastasis, it is not that useful a test because the remaining 39 who would not have developed metastasis would have undertaken chemotherapy.

In choosing the optimal cut-off level, the sensitivity (% true positive) data was most important since it gave a measure of how many patients that went on to develop metastasis had a +ve RAN score. If the sensitivity is lower than about 95%, a larger number of patients that were scored as Ran −ve would not have undertaken chemotherapy and would have gone on to develop metastasis.

The specificity (% true negative) data was less important since it related to patients that did not go on to develop metastasis. If the specificity was low, say 60%, a large number of patients (40 out of 100) that would have scored Ran +ve but would not have gone on to develop metastasis. Although that is not necessarily a problem since the clinician would not alter treatment for patients who scored Ran +ve, a high selectivity is preferred.

In view of these considerations, it can be seen that setting the cut-off level at 2 leads to a poor test because that level is too high. Only 23 of the 63 patients who scored Ran +ve were true positives, the test having low sensitivity (37.7%) simply because a large number of true positives had values below 2 which meant that they were classed as negative.

TABLE 9
The 2 × 2 contingency table when
Ran expression cut-off is equal to 1

| | Patients that did not go on to | Patients that went on to |
|---|---|---|
| | develop metastasis | develop metastasis |
| Ran − ve | 81 | 12 |
| Ran + ve | 96 | 49 |
| | Specificity = | Sensitivity = |
| | 81/146 | 49/61 |

It appears that a cut-off level of 1 is the best this data set notwithstanding that it does not have NPR and sensitivity which are sufficiently high for the assay of Ran levels to offer a credible predictive test for all types of breast cancer.

Table 9 shows a 2×2 contingency table for Ran expression in this set of patients when the RAN expression cut-off is equal to 1.

FIG. 3 shows a plot of distance metastasis free survival (DMFS) of Ran positive patients and Ran negative patients against time. As may be seen, the proportion of Ran negative patients surviving over 5 years is higher than the proportion of Ran positive patients. However, the proportion of Ran negative patients surviving over 5 years appears to drop towards 50% over 7½ years and to drop below 50% towards 10 years.

Example 1 shows that the addition of a second biomarker (MMP2) to the RanDx IHC test on tumour samples significantly improves both NPR and % sensitivity and to a level that is equivalent or better than Oncotype and significantly that this is for all breast cancer sub-types.

Accordingly, it is expected that a blood plasma assay for both Ran expression and MMP2 expression will afford clinicians a useful and accurate predictive test of the probability of a patient developing metastasis.

As may be expected from the standard genomic test Oncotype, if the probability is low the clinician will not give the patient chemotherapy and if the probability is high the clinician will advise that the patient receives chemotherapy.

Therefore, the blood test may of itself remove the burden of chemotherapy from those patients that do not need it, saving the health service considerable expenditure and providing the patient a better quality of life.

It may also allow the clinician to monitor patients post-surgery or during chemotherapy treatment. Changes over time in the level of Ran and/or MMP2 in the patient's blood may be indicative of a change in their risk of developing metastasis from dormant cancer cells. An increase in Ran or MMP2 blood level may indicate a change in the risk and the clinician may then prescribe a course of chemotherapy with the intention of reducing risk. Such a test is not presently available from conventional tests.

Note that references herein to Ran are references to Ran protein and that references to RAN are references to a RAN gene unless the context demands otherwise. References to MMP2 are references to a MMP2 protein or, where the context demands, MMP2 gene.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

1. A method for determining tumour status in a subject comprising the steps of:
(i) determining a quantitative value in a sample taken from a subject of a first biomarker selected from the group consisting of Ran protein, Ran binding protein 1, an active fragment of a Ran protein, a nucleic acid sequence encoding Ran, a nucleic acid sequence encoding Ran binding protein 1, a nucleic acid sequence encoding an active fragment of Ran and a nucleic acid sequence encoding an active fragment of Ran binding protein 1;
(ii) comparing the quantitative value of the first biomarker in the sample with a selected pre-determined threshold value of the first biomarker;
(iii) determining a quantitative value in a sample from the same subject of a second biomarker selected from the group consisting of MMP2 protein, an active fragment of MMP2 protein, a nucleic acid sequence encoding MMP2 and a nucleic acid sequence encoding an active fragment of MMP2;
(iv) comparing the quantitative value of the second biomarker in the sample with a selected pre-determined threshold value of the second biomarker;
wherein the quantitative values of the first marker and the second biomarkers in the sample as compared to their respective selected pre-determined threshold values indicate whether or not the tumour sample has invasive and/or metastatic potential.

2. A method according to claim 1 wherein the sample obtained for determination of a qualitative value of the first biomarker is the same or different sample as that used to determine the quantiataive value for the second biomarker.

3. A method according to claim 1 wherein the sample is selected from the group comprising a solid tumour biopsy, a liquid biopsy, a tumour cell, circulating tumour cells in blood, circulating tumour cells in blood plasma and circulating levels of biomarkers in a body fluid.

4. A method according to claim 1 wherein the selected predetermined threshold value of the first biomarker is at least between 0.5 and 5.0% of the sample reference value of the first biomarker.

5. A method according to claim 4 wherein the selected predetermined threshold value of the first biomarker is at least 1.0% of the sample reference value of the first biomarker.

6. A method according to claim 1 wherein the selected predetermined threshold value of the second biomarker is at least between 1.0 and 10.0% of the sample reference value of the second biomarker.

7. A method according to claim 6 wherein the selected predetermined threshold value of the second biomarker is at least 5.0% of the sample reference value of the second biomarker.

8. A method according to claim 4 wherein the sample reference value is the number of cells expressing the first and/or second biomarkers in subject having an invasive and/or metastatic tumour.

9. A method according to claim 8 wherein the number of cells is assessed immunohisto/immunocyto-chemically or other similar platform.

10. A method according to claim 4 wherein the sample reference value is the level of expression of the first and/or second biomarkers in subject having an invasive and/or metastatic tumour.

11. A method according to claim 10 wherein the level of expression of the first and second markers is assessed by ELISA, immunoprecipitation or immunoblotting or other protein detection platform.

12. A method according to claim 1 wherein, when the quantitative value of the first marker in the sample is lower than the selected predetermined threshold value for the first marker and the quantitative value of the second marker in the sample is lower than the selected predetermined threshold value for the second marker indicates that the tumour does not have invasive and/or metastatic potential.

13. A method according to claim 1 wherein, when the quantitative value of the first marker in the sample is above the selected predetermined threshold value for the first marker and the quantitative value of the second marker in the sample is above the selected predetermined threshold value for the second marker indicates that the tumour has invasive and/or metastatic potential.

14. A method according to claim 1 wherein, when the quantitative value of the first marker in the sample is lower than the selected predetermined threshold value for the first marker and the quantitative value of the second marker in the sample is above the selected predetermined threshold value for the second marker indicates that the tumour may have invasive and/or metastatic potential and requires further monitoring.

15. A method according to claim 1 wherein, when the quantitative value of the first marker in the sample is above the selected predetermined threshold value for the first marker and the quantitative value of the second marker in the sample is lower than the selected predetermined threshold value for the second marker indicates that the tumour may have invasive and/or metastatic potential and requires further monitoring.

16. A method according to claim 1 wherein the tumour is selected from the group comprising human breast cancer, and, in particular, an oestrogen receptor positive and human epidermal growth factor receptor 2 negative breast cancer cell or a triple receptor negative breast cancer (TNBC) cell.

17. A method according to claim 1 wherein the tumour status includes, monitoring for metastasis following surgery and/or during chemotherapy or radiotherapy, stratification of a group of subjects with cancer and/or predicting probability of survival/metastatic potential of a subject.

18. A kit comprising a first reagent and a second reagent for assessing respectively levels of a first marker and a second marker in a tumour sample or a blood plasma sample,
wherein the first marker is selected from the group consisting of Ran, Ran binding protein 1, an active fragment of a Ran protein, an active fragment of Ran binding protein 1, a nucleic acid sequence encoding Ran, a nucleic acid sequence encoding Ran binding protein 1, a nucleic acid sequence encoding an active fragment of Ran and a nucleic acid sequence encoding an active fragment of Ran binding protein 1; and
the second marker is selected from the group consisting of MMP2, an active fragment of MMP2 protein, a nucleic acid sequence encoding MMP2 and a nucleic acid sequence encoding an active fragment of MMP2.

19. A kit according to claim 18 for use in a method for determining whether a tumour in a subject has invasive and/or metastatic potential.

20. Use of a kit according to claim 18 for determining whether a tumour in a subject has invasive and/or metastatic potential.
